(19) United States
(12) Patent Application Publication
(10) Pub. No.: US 2022/0041710 A1
(43) Pub. Date: Feb. 10, 2022

(54) Treatment of brain ischemia-reperfusion injury

(71) Applicants: University of Zurich, Zurich (CH); XBiotech USA, Inc., Austin, TX (US)

(72) Inventors: Giovanni Guido Camici, Zurich (CH); Luca Liberale, Zurich (CH); Peter Libby, Cambridge, MA (US); John Simard, Austin, TX (US)

(21) Appl. No.: 17/508,973

(22) Filed: Oct. 23, 2021

Related U.S. Application Data
(63) Continuation-in-part of application No. PCT/US2020/030643, filed on Apr. 30, 2020.
(60) Provisional application No. 62/843,182, filed on May 3, 2019.

(51) Int. Cl. C07K 16/24; A61P 25/00

(52) U.S. Cl. CPC C07K 16/245; A61K 2039/505; A61P 25/00

(57) ABSTRACT

The sequelae of cerebral ischemia-reperfusion injury are reduced by administering to the subject a pharmaceutical composition that includes a pharmaceutically acceptable carrier and a therapeutically effective amount of an agent that selectively binds IL-1α.

### CROSS-REFERENCE TO RELATED APPLICATIONS

The present application is a bypass continuation-in-part under 35 U.S.C. 111(a) of international patent application number PCT/US2020/030643 filed on Apr. 30, 2020 which claims the priority of U.S. provisional patent application Ser. No. 62/843,182, entitled “Treatment of Brain Ischemia-Reperfusion Injury” and filed on May 3, 2019.

### STATEMENT AS TO FEDERALLY SPONSORED RESEARCH

Not applicable.

### FIELD OF THE INVENTION

The invention relates generally to the fields of medicine, neurology, and immunology. More particularly, the invention relates to the use of antibodies (Abs) which specifically bind interleukin-1α (IL-1α) to reduce various sequelae of ischemia-reperfusion injury to the central nervous system (e.g., the brain).

### BACKGROUND

Ischemic stroke is a major cause of death and disability. It is treated by removing the clot from the occluded vessel using tissue plasminogen activator or mechanical thrombectomy or endovascular therapy (EVT) such that blood flow to the affected tissue is restored. Restoration of blood flow, however, induces the release of excitotoxic neurotransmitters, intracellular Ca2+ accumulation, free radical damage, neuron apoptosis, neuroinflammation, and lipolysis which leads to ischemia-reperfusion injury. EVT is an attempt to minimize the duration of occlusion and associated exposure of affected brain tissue to ischemia and typically performed within 24 hours of the occlusion. However, in most cases EVT is not possible, such as when the occluded vessel cannot be accessed by an endovascular catheter. With these being the majority of cases, the blood clot affecting the occluded vessel naturally dissolves over time, as a result of normal hemostasis and endogenous clot dissolving mechanisms. An occluded artery causing stroke in the brain typically spontaneously dissolves within 24-72 hours from onset of the clot. The difference between EVT and natural recovery from a clot is therefore the duration of ischemia and timing of the reperfusion. Therefore, whether or not EVT is performed, therapies are needed to address ischemia reperfusion injury.

### SUMMARY

It has been discovered that a monoclonal antibody (mAb) that specifically binds IL-1α is useful for ameliorating the pathological sequelae that occur following cerebral ischemia-reperfusion injury.

Accordingly, described herein are methods of reducing one or more of the pathologic events that may occur after cerebral ischemia-reperfusion injury in a subject. These methods can include the step of administering to the subject a pharmaceutical composition including a pharmaceutically acceptable carrier and an amount of an agent that selectively binds IL-1α effective to reduce to edema, hemorrhagic transformation, intracranial pressure, breakdown of the blood-brain-barrier, the volume of the resulting infarct(s), and the resulting neurological deficit in the subject. The agent can be an anti-IL-1α antibody such as a monoclonal antibody (e.g., of the IgG1 isotype). The pharmaceutical composition can be administered to the subject by injection, subcutaneously, intravenously, intramuscularly, or intrathecally. In the method, the dose can be at least 50 mg (e.g., at least 50, 75, 100, 150, 200, 300, 400, 500, 600, 700, or 800 mg). Preferably, a first dose is administered within 1, 2, 3, 4, 5, 6 and up to 24 hours after observance of the first symptom of ischemic stroke or within 10, 20, 30, or 60 minutes after the subject seeks treatment by a medical professional. Thereafter, additional doses (e.g., at least 1, 2, 3, 4, 5, 6, 7, 8, 9, 10) may be administered to the subject, e.g., at about 20 min, 30 min, 45 min, 1 h, 2 h, 3 h, 6 h, 12 h, 1 d, 2 d, 3 d, 4 d, 5 d, 6 d, 7 d, 2 wk, 3 wk, or 4 wk after the previous administration.

Further described herein are methods of treating cerebral ischemia-reperfusion injury in a subject by administering to the subject an antibody that specifically binds interleukin-1α (IL-1α); methods of reducing the volume of the cerebral infarct that results from an occlusive stroke in a subject by administering to the subject an antibody that specifically binds IL-1α; methods of reducing the neurological deficit that results from an occlusive stroke in a subject by administering to the subject an antibody that specifically binds IL-1α; and methods of reducing the number of activated macrophages and/or the extent of inflammatory infiltrate and related insult in the ischemic penumbra resulting from an occlusive stroke in a subject by administering to the subject an antibody that specifically binds IL-1α. In the methods described above and here, the antibody that specifically binds IL-1α can be administered to the subject after the subject develops cerebral ischemia. In methods of reducing the volume of the cerebral infarct that results from an occlusive stroke in a subject, the volume of the cerebral infarct that results from the occlusive stroke in the subject can at least 10% (e.g., at least 20, 30, 40, or 50%) less than the volume of the cerebral infarct that would have resulted from the occlusive stroke if the subject was not administered the antibody that specifically binds IL-1α. Reduction in the ischemic stroke volume will invariably on average reduce the clinical impact of the infarct, reducing the symptoms and long term consequence of an acute ischemic stroke.

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of medical terms can be found in Stedman's Medical Dictionary, 27^{th} Edition, Lippincott, Williams & Wilkins, 2000.

As used herein, an “antibody” or “Ab” is an immunoglobulin (Ig), a solution of identical or heterogeneous Igs, or a mixture of Igs. An “Ab” can also refer to fragments and engineered versions of Igs such as Fab, Fab′, and F(ab′)₂ fragments; and scFv's, heteroconjugate Abs, and similar artificial molecules that employ Ig-derived CDRs to impart antigen specificity. A “monoclonal antibody” or “mAb” is an Ab expressed by one clonal B cell line or a population of Ab molecules that contains only one species of an antigen binding site capable of immunoreacting with a particular epitope of a particular antigen. A “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is associated naturally or during the cell culture manufacturing process. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.”

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹⁰, 10¹¹, or 10¹² liters/mole for that other molecule. An Ab that “selectively binds” a first molecule specifically binds the first molecule at a first epitope but does not specifically bind other molecules that do not have the first epitope. For example, an Ab which selectively binds IL-1alpha specifically binds an epitope on IL-1alpha but does not specifically bind IL-1beta (which does not have the epitope).

A “therapeutically effective amount” is an amount which is capable of producing a medically desirable effect in a treated animal or human (e.g., amelioration or prevention of a disease or symptom of a disease).

Although methods and materials similar or equivalent to those described herein can be used in the practice or testing of the present invention, suitable methods and materials are described below. All applications and publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a graph showing tissue IL-1α levels in the contralateral hemisphere versus the ipsilateral hemisphere of brains in mice administered an isotype control antibody after transient middle cerebral artery occlusion (tMCAO) was performed.

FIG. 2 is a graph showing the reduction in infarct volume in subjects treated with an anti-IL-1α antibody compared to subjects treated with an isotype control antibody.

FIG. 3 is a graph showing the improvement in Bederson Index scores in subjects treated with an anti-IL-1α antibody compared to subjects treated with an isotype control antibody.

FIG. 4 is a graph showing the reduction Latency to Fall time in the RotaRod test in subjects treated with an anti-IL-1α antibody compared to subjects treated with an isotype control antibody.

FIG. 5 is a series of photomicrographs (top) showing expression of P-selectin and YE-cadherin in the blood brain barrier of tMCAO-treated mice administered an anti-IL-1α antibody or an isotype control antibody; and a graph (bottom) showing lower P-selectin expression in subjects treated with an anti-IL-1α antibody compared to subjects treated with an isotype control antibody.

FIG. 6 is a series of photomicrographs (top) showing expression of ICAM-1 expression in the brain endothelium of tMCAO-treated mice administered an anti-IL-1α antibody or an isotype control antibody; and a graph (bottom) showing lower ICAM-1 expression in subjects treated with an anti-IL-1α antibody compared to subjects treated with an isotype control antibody.

FIG. 7 is a series of photomicrographs (top) showing expression of VCAM-1 expression in the brain endothelium of tMCAO-treated mice administered an anti-IL-1α antibody or an isotype control antibody; and a graph (bottom) showing lower VCAM-1 expression in subjects treated with an anti-IL-1α antibody compared to subjects treated with an isotype control antibody.

FIG. 8 is a series of photomicrographs (top) showing decreased numbers of activated macrophages in the stroke area of tMCAO-treated mice administered an anti-IL-1α antibody compared to those administered an isotype control; and a graph (bottom) showing the same.

FIG. 9 is a series of photomicrographs (top) showing lower MMP9 expression in the penumbra area of tMCAO-treated mice administered an anti-IL-1α antibody compared to those administered an isotype control; and a graph (bottom) showing the same.

### DETAILED DESCRIPTION

Described herein are compositions and methods for reducing one or more sequelae of cerebral ischemia-reperfusion injury in a subject. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. General methods of medical treatment are described in McPhee and Papadakis, Current Medical Diagnosis and Treatment 2010, 49th Edition, McGraw-Hill Medical, 2010; and Fauci et al., Harrison's Principles of Internal Medicine, 17^{th} Edition, McGraw-Hill Professional, 2008. Methods in neurology are described in Daroff R., Bradley's Neurology in Clinical Practice, 2-Volume Set 7th Edition, Elsevier, 2015.

### Treatment

The compositions described herein are useful for treating cerebral ischemia-reperfusion injury in a mammalian subject by administering to the subject a pharmaceutical composition including an amount of an anti-IL-1α Ab effective to improve at least one characteristic of the condition in the subject (e.g., edema, hemorrhagic transformation, intracranial pressure, breakdown of the blood-brain-barrier, inflammatory cell infiltration of ischemic parenchyma, infarct volume, and the resulting neurological deficit in the subject). Successful treatment of cerebral ischemia-reperfusion injury can be evaluated according to established methods. These include neurological examination, computed tomography, magnetic resonance imaging, and cerebral angiography. Improvement can be assessed as scoring at least 10% (e.g., at least 10, 20, 30, 40, 50, 60, or 70%) better on tests used to evaluate the sequelae of cerebral ischemia-reperfusion injury at a given time after onset of the injury (e.g., 1, 2, 3, 4, 5, 7, 10, or 30 days; or 1, 2, 3, 4, 5, 6, 12 or 24 months after onset of the injury) compared to if the subject was not administered the effective amount of the anti-IL-1α Ab (e.g., compared to genetically matched subject or as extrapolated from historical data of subjects having a similar injury).

Cerebral infarct volume subsequent to a cerebral ischemia-reperfusion injury can be determined in living subjects by magnetic resonance imaging (MRI) as described for example in Lovblad et al., Ann Neurol. 42:164-170, 1997. Preferably this is performed when the final infarct volume is reached (e.g., at least 30 days after the injury). The quantity and/or quality of neurological deficit that results from a cerebral ischemia-reperfusion injury in a subject can be determined by known methods such as the National Institutes of Health Stroke Scale (NIHSS), which measures neurologic impairment using a 15-item scale (table 1) or the Canadian Neurological Scale. The number of activated macrophages/microglia in the ischemic penumbra of a brain lesion that results from a cerebral ischemia-reperfusion injury can be assessed in live subjects by MRI where ultrasmall superparamagnetic iron oxide (USPIO) are used as macrophages/microglia-specific contrast agents or other known methods.

The subject can be a mammal such as a human being, a rodent, a cat, a dog, a horse, a sheep, or a pig that has suffered, is suffering from, or is at risk of developing cerebral ischemia (e.g., ischemic stroke, transient ischemic attack, or subarachnoid hemorrhage) including, human beings. Human subjects might be male, female, adults, children, seniors (65 and older), and those with other diseases or risk factors for cerebral ischemia (e.g., hypertension, diabetes, heart disease, race/ethnicity, personal or family history of cerebral ischemia, brain aneurysms, and/or brain arteriovenous malformations). As a non-limiting example, the subject can be a human being diagnosed with cerebral vessel occlusion or one having transient ischemic attacks. The subject can also be a human being who has been administered tissue plasminogen activator (TPA) (e.g., following being diagnosed with acute cerebral vessel occlusion). The subject may also be administered endovascular therapy alone or in combination with TPA to relieve the occlusion. The occlusion will anyway be expected to dissolve in many cases without intervention of any kind, due to natural mechanisms of thrombus resorption, and administering anti-IL-1a therapy can be expected to reduce sequelae, such as brain injury and neurological dysfunction, in the event it is administered to subjects who otherwise have no intervention; administering the anti-IL-1a is expected to reduce hypoxia-related inflammatory insult and post-hypoxia related reperfusion injury. The initial dose of the agent that binds IL-1α can be administered within 1, 2, 3, 4, 5, 6, 7, 8, 9, 10, 11, 12, 13, 14, 15, 16, 17, 18, 19, 20, 24, 36, or 48 h of the onset of symptoms of ischemic stroke. For a subject at high risk for developing an ischemic stroke (e.g., subject experiencing transient ischemic attacks or having a thrombosis), the agent that binds IL-1α can be administered prophylactically, with a frequency of once a day, or once every 2, 3, 4, 5, 6, 7, or 14 days until the risk is decreased (e.g., transient ischemic attacks stop or the thrombosis is cleared). It is preferred to treat subjects who have developed a human anti-human antibody response due to prior administration of therapeutic antibodies with an anti-IL-1α Ab that is a true human Ab (e.g., one that is naturally expressed in a human subject).

### Antibodies and Other Agents that Target IL-1α

Any suitable type of Ab that specifically reduces one or more sequelae of cerebral ischemia-reperfusion injury in a subject might be used in the methods described herein. For example, the anti-IL-1α Ab used might be mAb, a polyclonal Ab, a mixture of mAbs, or an Ab fragment or engineered Ab-like molecule such as an scFv. The Ka of the Ab is preferably at least 1×10⁹ M⁻¹ or greater (e.g., greater than 9×10¹⁰ M⁻¹, 8×10¹⁰ M⁻¹, 7×10¹⁰ M⁻¹, 6×10¹⁰ M⁻¹, 5×10¹⁰ M⁻¹, 4×10¹⁰ M⁻¹, 3×10¹⁰ M⁻¹, 2×10¹⁰ M⁻¹, or 1×10¹⁰ M⁻¹). In a preferred embodiment, the Ab is a fully human mAb that includes (i) an antigen-binding variable region that exhibits very high binding affinity (e.g., at least nano or picomolar) for human IL-1α and (ii) a constant region. The human Ab is preferably an IgG1, although it might be of a different isotype such as IgM, IgA, or IgE, or subclass such as IgG2, IgG3, or IgG4. Useful mAbs include those that neutralize IL-1α (e.g., those that prevent IL-1α from binding an IL-1α receptor).

Because B lymphocytes which express Ig specific for human IL-1α occur naturally in human beings, a presently preferred method for raising mAbs is to first isolate such a B lymphocyte from a subject and then immortalize it so that it can be continuously replicated in culture. Subjects lacking large numbers of naturally occurring B lymphocytes which express Ig specific for human IL-1α may be immunized with one or more human IL-1α antigens to increase the number of such B lymphocytes. Human mAbs are prepared by immortalizing a human Ab secreting cell (e.g., a human plasma cell). See, e.g., U.S. Pat. No. 4,634,664.

In an exemplary method, one or more (e.g., 5, 10, 25, 50, 100, 1000, or more) human subjects are screened for the presence of such human IL-1α-specific Ab in their blood. Those subjects that express the desired Ab can then be used as B lymphocyte donors. In one possible method, peripheral blood is obtained from a human donor that possesses B lymphocytes that express human IL-1α-specific Ab. Such B lymphocytes are then isolated from the blood sample, e.g., by cells sorting (e.g., fluorescence activated cell sorting, “FACS”; or magnetic bead cell sorting) to select B lymphocytes expressing human IL-1α-specific Ig. These cells can then be immortalized by viral transformation (e.g., using EBV) or by fusion to another immortalized cell such as a human myeloma according to known techniques. The B lymphocytes within this population that express Ig specific for human IL-1α can then be isolated by limiting dilution methods (e.g., cells in wells of a microtiter plate that are positive for Ig specific for human IL-1α are selected and subcultured, and the process repeated until a desired clonal line can be isolated). See, e.g., Goding, MAbs: Principles and Practice, pp. 59-103, Academic Press, 1986. Those clonal cell lines that express Ig having at least nanomolar or picomolar binding affinities for human IL-1α are preferred. MAbs secreted by these clonal cell lines can be purified from the culture medium or a bodily fluid (e.g., ascites) by conventional Ig purification procedures such as salt cuts, size exclusion, ion exchange separation, and affinity chromatography.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an mAb specific for human IL-1α might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H₂L₂ configuration, the genes encoding each may be separately isolated and expressed in different vectors.

Although generally less preferred due to the greater likelihood that a subject will develop an anti-Ab response, chimeric mAbs (e.g., “humanized” mAbs), which are antigen-binding molecules having different portions derived from different animal species (e.g., variable region of a mouse Ig fused to the constant region of a human Ig), might be used. Such chimeric Abs can be prepared by methods known in the art. See, e.g., Morrison et al., Proc. Nat'l. Acad. Sci. USA, 81:6851, 1984; Neuberger et al., Nature, 312:604, 1984; Takeda et al., Nature, 314:452, 1984. Similarly, Abs can be humanized by methods known in the art. For example, mAbs with a desired binding specificity can be humanized by various vendors or as described in U.S. Pat. Nos. 5,693,762; 5,530,101; or 5,585,089.

The mAbs described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992 Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water soluble polymer such as polyethylene glycol or a carbon nanotube (See, e.g., Kam et al., Proc. Natl. Acad. Sci. USA 102: 11600-11605, 2005). See, U.S. patent application Ser. No. 11/754,899.

Preferably, to ensure that high titers of human IL-1α-specific mAb can be administered to a subject with minimal adverse effects, the mAb compositions should be at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions might include only a single type of mAb (i.e., one produced from a single clonal B lymphocyte line) or might include a mixture of two or more (e.g., 2, 3, 4, 5, 6, 7, 8, 9, 10 or more) different types of mAbs.

While the IL-1α specific Abs described above are preferred for use, in some cases, other agents that specifically target IL-1α might be used so long as their administration leads to a reduction in one or more sequelae of cerebral ischemia-reperfusion injury in a subject as used in the methods described herein. Because some IL-1α specific Abs have been shown to block the action of IL-1α by preventing its interaction with the IL-1 receptor (IL-1R1), based on this mechanism of action in treating various pathological conditions, other Abs or non-Ab agents that also block IL-1c from interacting with IL-1R1 could also be used (e.g., other anti-IL-1a Abs or anti-IL-1R1 Abs which block IL-1c from interacting with IL-1R1). These Abs can be made according to the methods described above. Non-Ab agents might include vaccines that cause the production of anti-IL-1α Abs which block IL-1α from interacting with IL-1R1, proteins or peptides that bind IL-1α and block IL-1α from interacting with IL-1R1, and small organic molecules which specifically target IL-1α and block IL-1α from interacting with IL-1R1. Those that do not specifically bind IL-1β are preferred. Whether a particular agent is able to reduce one or more sequelae of cerebral ischemia-reperfusion injury in a subject can be determined by the methods described in the Examples section below.

### Pharmaceutical Compositions and Methods

The anti-IL-1α Ab compositions (and other agents that specifically target IL-1α) may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent.

The Ab compositions may be administered to animals or humans by any suitable technique. Typically, such administration will be parenteral (e.g., intravenous, subcutaneous, intramuscular, intrathecal, or intraperitoneal introduction). The compositions may also be administered directly to the target site (e.g., the brain or lesion site) by, for example, application using a catheter using X-ray guidance. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-IL-1α Ab compositions is an amount which shows clinical efficacy in patients as measured by a reduction in one or more sequelae of cerebral ischemia-reperfusion injury. As is well known in the medical arts, dosage for any one animal or human depends on many factors, including the subject's size, body surface area, age, the particular composition to be administered, sex, time and route of administration, general health, and other drugs being administered concurrently. Preferred doses range from about 3 to 100 (e.g., 2, 3, 4, 5, 6, 7, 8, 9, 10, 11, 12, 13, 14, 15, 16, 17, 18, 19, 20, 30, 40, 50, 60, 70, 80, 90, or 100) mg/kg body weight. In some cases, a single dose may be effective. In other cases, doses may be given repeatedly, e.g., 20 min, 30 min, 45 min, 1 h, 2 h, 3 h, 6 h, 12 h, 1 d, 2 d, 3 d, 4 d, 5 d, 6 d, 7 d, 2 wk, 3 wk, or 4 wk after the previous administration.

### EXAMPLES

### Example 1

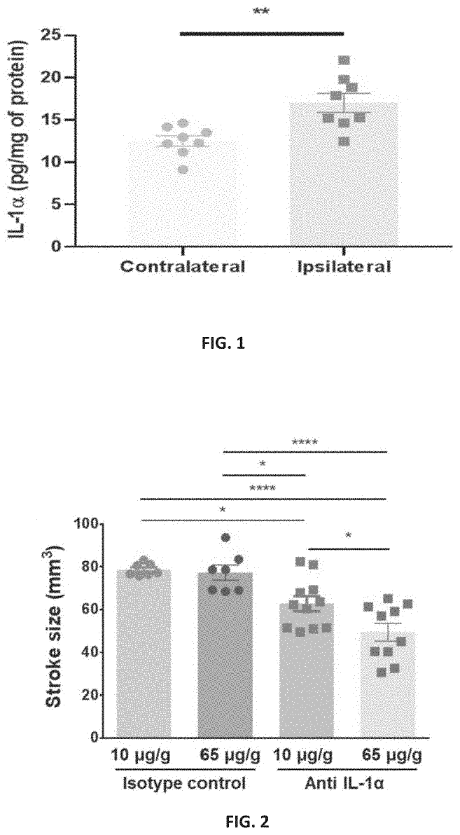

12-week-old male C57BL/6 Wild-type (WT) mice underwent transient middle cerebral artery occlusion (tMCAO) for 45 minutes. To induce ischemia/reperfusion (I/R) brain injury, transient middle cerebral artery occlusion (tMCAO) was performed as shown in FIG. 1. Briefly, mice were anaesthetized using isoflurane 3% and 1.5% for induction and maintenance, respectively. For analgesia, buprenorphine HCl was infiltrated at the incision side (0.1 mg/Kg). Ischemia was induced by inserting a 6-0 silicone-coated filament into the common carotid artery until the origin of the left MCA after the dissection of common, internal and external carotid arteries. Next, mice randomly received either mouse anti-mouse IL-1α antibody (i.e., Flo1-2a) or isotype control at different dosages (10 or 65 μg/g). IL-1α inhibition was performed after the ischemic event upon reperfusion as it would be the case in patients presenting to the emergency care unit and eligible for thrombolytic therapy. More particularly, animals were randomized to receive either anti-IL-1α antibody or appropriate isotype control antibody via tail vein injection at the time of filament retraction (i.e. beginning of reperfusion period).

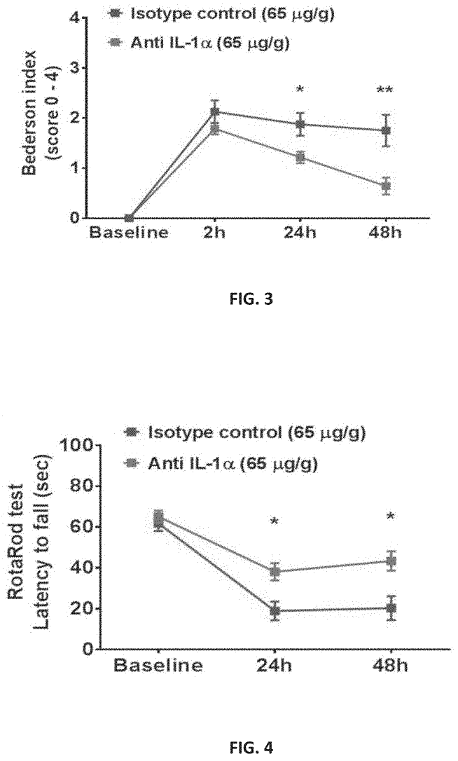

Forty-eight hours after tMCAO, stroke (infarct) volume was determined by 2,3,5-triphenyltetrazolium chloride (TTC) staining and neurological deficit by a four-point scale neurological score (Bederson Index; Bederson et al., Stroke. 1986; 17:472-476) as well as by RotaRod test. In control (isotype-matched) antibody-treated mice exposed to tMCAO, tissue IL-1α levels rose in the ipsilateral hemisphere underscoring the pathophysiological relevance of this cytokine for stroke (FIG. 1). After 48 h of reperfusion, mice treated with the lower dose of the anti-IL-1α antibody showed a minor reduction in infarct volume, as assessed by TTC staining, although post-stroke neurological deficit did not improve (not shown). Treatment with the higher dose of the anti-IL-1α antibody reduced stroke size by 36% compare to isotype control, and improved neurological performance as determined by Bederson and RotaRod tests (FIGS. 2-4).

Post-ischemic blood-brain barrier (BBB) damage importantly influences stroke outcome. Immunohistochemical analysis of IgG extravasation demonstrated a slight (not statistically significant) trend towards increased BBB permeability in the anti-IL-1α-treated animals. Similarly, rates of hemorrhagic transformation as assessed macroscopically did not differ among the groups. Likewise, the endothelial expression of occludin, claudin 5 and VE-cadherin-regulators of paracellular BBB permeability—did not differ between the treated and the control groups.

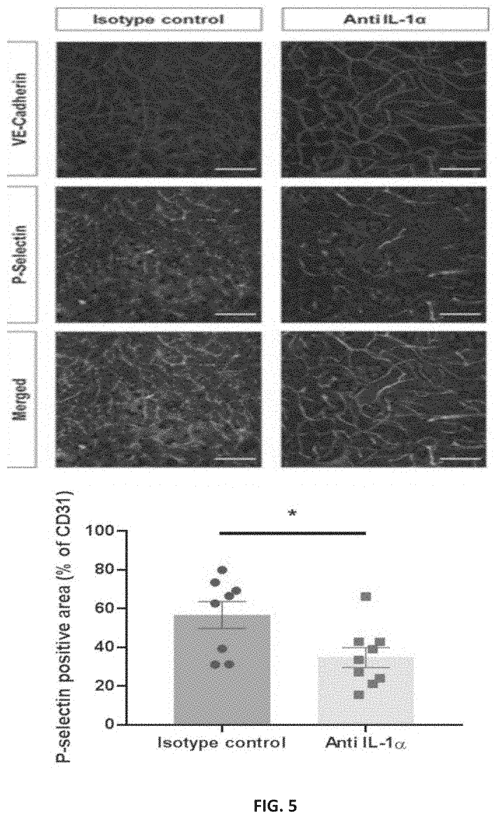

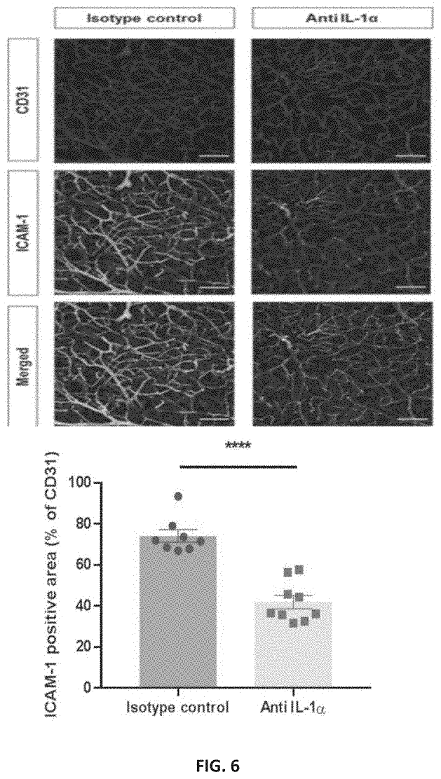

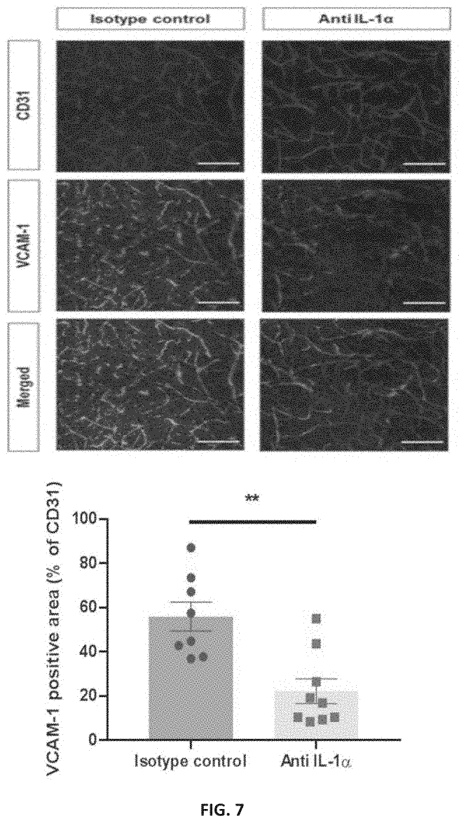

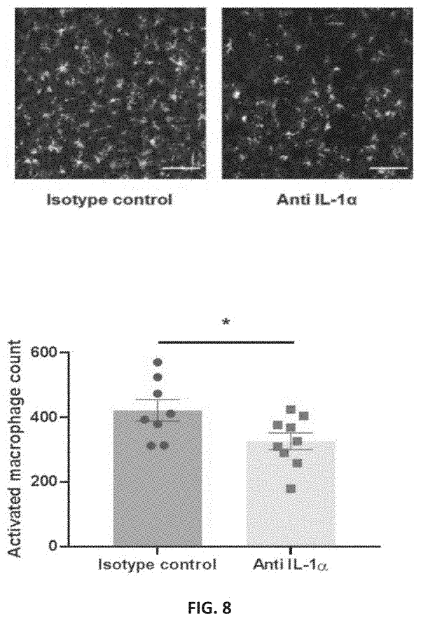

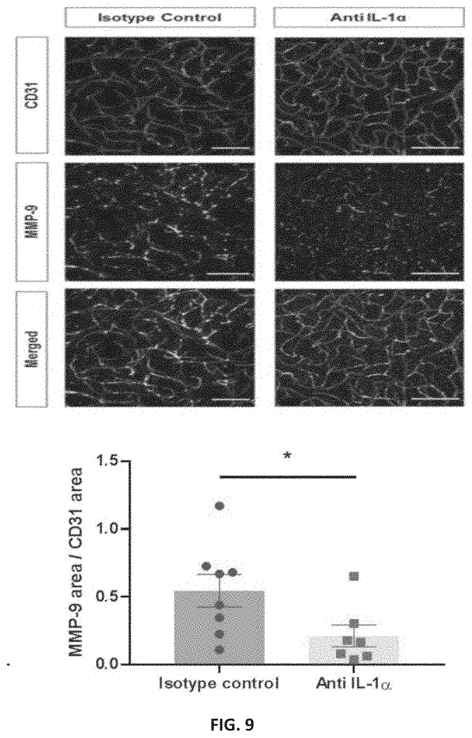

Following stroke, the local rise of damage-associated molecular patterns and other inflammatory mediators recruit circulating leukocytes to the damage site and facilitate their effector functions. Leukocyte migration depends on a complex pattern of adhesion molecules expressed by both brain microvascular endothelial cells and white blood cells including selectins, adhesion molecules of the immunoglobulin superfamily and integrins. Confocal microscopy of the penumbra area demonstrated decreased endothelial expression of P-selectin, ICAM-1 and VCAM-1 in animals treated with the IL-1α inhibitory antibody as compared to control littermates (FIGS. 5-7). Following ischemia, activation of resident immune cells of the brain (i.e. microglia) as well as infiltration of monocytes from circulating pool contributes importantly to brain tissue damage. Post-ischemic IL-1α neutralization significantly decreased numbers of activated macrophages in the stroke area compared to control mice as assessed by Iba-1 immunostaining (FIG. 8). Upon activation, macrophages secrete several pro-inflammatory mediators such as TNF-α, ILs and MMPs, thereby exacerbating cerebral parenchymal damage. Among these, MMP9 can exert direct neurotoxic effects. In line with the Iba-1 data, further immunohistochemical analysis of the penumbra area revealed reduced MMP9 tissue levels in animals receiving the IL-1α neutralizing antibody as compared to controls (FIG. 9).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cerebral ischemia-reperfusion injury in a subject that has experienced a cerebral ischemic event, the method comprising the step of administering to the subject an antibody that specifically binds interleukin-1α (IL-1α) after the subject develops cerebral ischemia.

2. The method of claim 1, wherein the cerebral ischemic event causes the subject to develop a cerebral infarct, and the step of administering the antibody to the subject reduces the volume of the cerebral infarct compared to the volume of the cerebral infarct that would have developed if the subject was not administered the antibody.

3. The method of claim 2, wherein the volume of the cerebral infarct that results from the cerebral ischemic event in the subject is at least 20% less than the volume of the cerebral infarct that would have resulted from the occlusive stroke if the subject was not administered the antibody.

4. The method of claim 1, wherein the cerebral ischemic event causes the subject to develop a neurological deficit, and the step of administering the antibody to the subject reduces the magnitude of the neurological deficit compared to the magnitude of the neurological deficit that would have developed if the subject was not administered the antibody.

5. The method of claim 1, wherein the cerebral ischemic event leads to the presence of activated macrophages in the ischemic penumbra of a brain lesion that results from the cerebral ischemic event in a subject, and the step of administering the antibody to the subject reduces the number of activated macrophages present in the ischemic penumbra of the brain lesion compared to the number of activated macrophages that would have been present in the ischemic penumbra of the brain lesion if the subject was not administered the antibody.
